(19) United States
(12) Patent Application Publication
(10) Pub. No.: US 2003/0162769 A1
(43) Pub. Date: Aug. 28, 2003

(54) Composition and method for treating vulvodynia

(76) Inventor: W. Jerry Easterling, San Antonio, TX (US)

Correspondence Address: DAVID G. HENRY, 900 Washington Avenue, P.O. Box 1470, Waco, TX, 76701, US

(21) Appl. No.: 10/083,625

(22) Filed: Feb. 26, 2002

(51) Int. Cl. A61K 31/554

(52) U.S. Cl. 514/211.07

(57) ABSTRACT

The invention is of a medicament and associated treatment involving the use of such medicament for the treatment of vulvodynia. The medicament is in the dosage form of a vaginal suppository, and the primary active ingredient is a calcium antagonist—Diltiazem Hydrochloride in the preferred embodiment.

### BACKGROUND OF THE INVENTION

1. Field of the Invention

The present invention relates to medical treatments pertaining to vulvodynia.

2. Background Information

Vulvodynia is characterized by unexplained vulvar pain that can cause physical disability, sexual dysfunction, limitation of normal daily activities, and psychological difficulties.

The problem often becomes chronic, lasting for years. There are four basic types of vulvodynia: (1) vulvar vestibulitis (2) dysesthetic vulvodynia (3) vulvar dermatoses and (4) cyclic vulvovaginitis. Many patients are misdiagnosed or not diagnoses at all. Pain is not always accompanied by visible tissue changes, thereby complicating an accurate diagnosis. Vulvar vestibulitis and dysesthetic vulvodynia are the most common.

The etiology of the disease is unknown. However, it has been hypothesized that viral, fungal and bacterial assaults, allergic reactions, and an autoimmune response to the body's own chemistry may play a role in the disease process. Irritation of the muscles that support the uterus, bladder and rectum as well as irritation of the nerves of the vulva tissue may result in the painful symptoms associated with Vulvodynia.

Empirical evidence indicates that approximately fifteen percent of the adult female population may suffer from Vulvodynia at sometime during their lifetime. Approximately seventy percent of women with vulvodynia are white, have fair complexion, and are of child bearing age. A study published in the Journal of Urology in May of 1997 suggested that ten percent of women with interstitial cystitis also have symptoms of Vulvodynia.

Many patients experience difficulty is walking, sensitivity to clothing touching the vaginal area, difficulty with sexual activities due to pain, difficulty in sitting for long periods, and mild to intense pain described as burning, stinging, or itching.

Treatments for vulvodynia include oral medications such as antihistamines, tricyclic antidepressants; topical estrogens; and anticonvulsants; physical therapy and biofeedback; Interferon intralesional injections; low oxalate diet; oral calcium citrate; laser therapy; and surgery. Laser and surgical treatment complications include hematoma, wound dehiscence, uneven healing, and stenosis of the Bartholin's duct with cyst formation. There is no known cure for Vulvodynia.

### SUMMARY OF THE INVENTION

It is an object of the present invention to provide a novel medicament to be used for the treatment of Vulvodynia.

It is another object of the present invention to provide a novel medicament and unobvious medicament for the treatment of Vulvodynia, which medicament is more effective than existing means for treatment.

In satisfaction of these and related objectives, Applicant's present invention provides the vaginal application of a calcium channel blocker agent (preferably in suppository form) and associated methodology for use thereof, through the use of which Vulvodynia may be effectively, noninvasively, cost effectively, and painlessly treated.

### DETAILED DESCRIPTION OF THE PREFERRED EMBODIMENT

The preferred embodiment of the medicament of the present invention is a vaginal suppository which has demonstrated relief from the symptoms of Vulvodynia in as little as ten days of treatment.

In the preferred embodiment, the primary active ingredient of the vaginal suppository is Diltiazem Hydrochloride, USP, a benzothiazepine calcium channel blocker. However, it should be understood that other calcium channel blockers (topically applied, may provide similar relief. Others include Verapamil, a diphenylalkylamine, dihydropyridines, and the fast sodium inward channel inhibitor, Bepridil.

The preferred Diltiazem-based vaginal suppository formulation follows:

| | |
|---|---|
| Diltiazem 50 mg vaginal Suppository | 30 each |
| Diltiazem Hydrochloride, USP | 1.50 Gm |
| Silica Gel, micronized | 0.45 Gm |
| Base MBK (Fatty Acid) | 32.85 Gm |

Melt the Base MBK at 50 degrees Centigrade. Triturate the Diltiazem with the Silica Gel. Using a wire mesh strainer, sprinkle the powdered mixture into the melted base with stirring. Remove from heat and continue stirring until a uniform suspension exists. Pour into suppository shells and allow to cool at room temperature. Heat seal the open ends of the suppository shells. STORE IN REFRIGERATOR at 4 degrees Centigrade.

The recommended single dose of the Diltiazem Vaginal Suppository contains 50 mg of Diltiazem and is contained in 1.16 Gm of the preferred embodiment of the suppository.

Packaging in which the suppositories are dispensed to patients should be labeled with the following legend: STORE IN REFRIGERATOR.

The patient is to insert vaginally one suppository once or twice daily, depending on patient response as measured by the patient's physician. During treatment, the patient's progress should be evaluated by the physician at least every thirty days.

It should be noted that Diltiazem is commonly given orally to treat hypertension or cardiac arrhythmias. Patients should be counseled to report any side effect that could relate to blood pressure changes or noticeable heart rate changes. Any vaginal mucosa irritation should also be immediately reported.

It is unclear how the medicament of the present invention works to relieve the symptoms of Vulvodynia. The inventor believes that upon successful absorption of the Diltiazem into the vaginal mucosa, that the calcium channel blocking properties of the Diltiazem may exert an antivasoconstrictor activity or initiate a non-vascular process such as serotonin release or serotonin and histamine receptor blockade. The inventor also believes that after repeated use of the medicament, that a tissue remodeling of damaged or scarred tissue may occur, resulting in a healthier tissue accompanied by resolution of symptoms. The tissue remodeling process consists of the calcium channel blocker medication initiating the production of collagenase within the diseased tissue which initiates the remodeling process while the drug also causes a reduction in the production of fibroblasts associated with the production of scar tissue.

The medicament of the present invention has also shown efficacy in the treatment of symptoms associated with Interstitial Cystitis when used vaginally once or twice a day. This indicates that the present medicament has application well beyond the treatment of Vulvodynia, and promises relief of symptoms in any disease of similar mechanisms or physical manifestations like those of Vulvodynia.

Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for the treatment of vulvodynia comrpising:
a therapeutic dosage of a calcium channel blocker agent;
a vaginal suppository member in which said therapeutic dosage form is encased.

2. The medicament of claim 1 wherein said calcium channel blocker agent is a benzothiazepine calcium channel blocker.

3. The medicament of claim 2 wherein said calcium channel blocker agent is Diltiazem Hydrochloride.

4. A method for treating vulvodynia comprising the steps of:
selecting a dosage form comprising:
a therapeutic dosage of a calcium channel blocker agent;
a vaginal suppository member in which said therapeutic agent is encased;
administering said dosage form at least once daily until symptoms are reversed.

5. The method of claim 4 wherein said calcium channel blocker agent is a benzothiazepine calcium channel blocker.

6. The method of claim 5 wherein said calcium channel blocker agent is Diltiazem Hydrochloride.

7. The medicament of claim 3 wherein said therapeutic dosage is approximately 50 mg.
